(19) United States
(12) Patent Application Publication
(10) Pub. No.: US 2026/0174714 A1
(43) Pub. Date: Jun. 25, 2026

(54) NONHUMAN ANIMAL AND USE THEREOF

(71) Applicant: THE JIKEI UNIVERSITY, Tokyo (JP)

(72) Inventors: Shuichiro Yamanaka, Tokyo (JP); Kenji Matsui, Tokyo (JP); Takashi Yokoo, Tokyo (JP)

(73) Assignee: THE JIKEI UNIVERSITY, Tokyo (JP)

(21) Appl. No.: 18/854,317

(22) PCT Filed: Mar. 31, 2023
(86) PCT No.: PCT/JP2023/013642

§ 371 Date: Oct. 4, 2024

(30) Foreign Application Priority Data
Apr. 7, 2022 (JP) 2022-063993

(51) Int. Cl. A61K 31/19; A61K 9/70

(52) U.S. Cl. CPC A61K 31/19; A61K 9/70

(57) ABSTRACT

A nonhuman animal having a chimeric organ including a foreign cell in a body thereof.

### TECHNICAL FIELD

The present invention relates to a nonhuman animal and a use thereof.

### BACKGROUND ART

An acute kidney injury (also referred to as an AKI) accounts for about 15% of all hospitalized patients (see, for example, Non Patent Document 1), and even AKI at Stage 1 significantly increases any of a mortality, a hospitalization period, and a medical expense (see, for example, Non Patent Document 2).

Among those, a drug-induced kidney injury accounts for about 20% of AKI's (see, for example, Non Patent Document 3), and also gives an influence on treatment options for severe diseases, such as forcing patients to refrain from using anticancer drugs, antibiotics, or the like which can cause kidney injury.

In addition, since the causes of the diseases are iatrogenic, there is also a problem from the viewpoint of medical safety. However, the treatment thereof mainly involves include reducing or discontinuing suspected drugs, supplementing with saline, and administering steroids, and development of a specific treatment method has not progressed (see, for example, Non Patent Document 3).

In drug discovery, clinical trials using human bodies are performed after adverse events and therapeutic effects are confirmed through preclinical trials using rodents. However, reactions in the rodents do not completely match those in the human bodies, and nephrotoxicity that could not be confirmed in preclinical trials is pointed out for the first time in the clinical trial, resulting in many cases of the drug development being interrupted, resulting in huge financial and time losses (see, for example, Non Patent Document 4). It has also been reported that 19% of new drugs are interrupted due to nephrotoxicity at a stage of clinical trials (see, for example, Non Patent Document 5).

### CITATION LIST

### Non Patent Documents

Non Patent Document 1: Iwagami M, Moriya H, Doi K, Yasunaga H, Isshiki R, SatoI, et al. Seasonality of acute kidney injury incidence and mortality among hospitalized patients. Nephrology Dialysis Transplantation. 2018; 33(8): 1354-62.
Non Patent Document 2: Collister D, Pannu N, Ye F, James M, Hemmelgarn B, ChuiB, et al. Health Care Costs Associated with AKI. Clinical Journal of the American Society of Nephrology. 2017; 12(11): 1733-43.
Non Patent Document 3: Awdishu L, Mehta R L. The 6R's of drug induced nephrotoxicity. BMC Nephrology. 2017; 18(1).
Non Patent Document 4: Troth S P, Simutis F, Friedman G S, Todd S, Sistare F D. Kidney Safety Assessment: Current Practices in Drug Development. Seminars in Nephrology. 2019; 39(2): 120-31.
Non Patent Document 5: Tiong H Y, Huang P, Xiong S, Li Y, Vathsala A, Zink D. Drug-induced nephrotoxicity: clinical impact and preclinical in vitro models. Mol Pharm. 2014; 11(7): 1933-48.

### SUMMARY OF INVENTION

### Technical Problem

Given such a background, there is a high need for accurately detecting nephrotoxicity at a preclinical trial stage in the development of novel drugs, and for developing specific treatment methods for kidney injuries such as drug-induced kidney injury, and it is considered that a human nephrotoxicity evaluation model using rodent animals such as chimeric mice and chimeric rats equipped with human kidneys can meet this need.

Therefore, an object of the present invention is to provide a nonhuman animal that is suitably used for developing a novel drug and developing a treatment method specific for a disease, a method for producing the nonhuman animal, and a drug evaluation method.

### Solution to Problem

The present invention includes the following aspects.
[1] A nonhuman animal having a chimeric organ including a foreign cell in a body of the nonhuman animal.
[2] The nonhuman animal according to [1], in which the foreign cell is a rodent cell, a human cell, a cell derived from an individual patient, a labeled cell, a genome-edited cell, or a cell obtained by combining these cells.
[3] The nonhuman animal according to [1], in which the nonhuman animal is a pathological model.
[4] The nonhuman animal according to [1], in which the nonhuman animal is a drug administration model.
[5] A method for producing the nonhuman animal according to any one of [1] to [4], the production method including: a step of injecting a foreign cell into a kidney of a neonate.
[6] The production method according to [5], in which the foreign cell is a precursor cell obtained by differentiating a pluripotent stem cell derived from a hereditary disease patient.
[7] The production method according to [5], in which the foreign cell is a cell in which at least one target gene is edited.
[8] The production method according to [7], in which the foreign cell is a cell having a labeled protein associated with the edited target gene.
[9] A drug evaluation method including: a step of administering a drug to the nonhuman animal according to any one of [1] to [4]; and a step of acquiring a single cell derived from a foreign cell from a chimeric organ after the drug administration, and evaluating the single cell.

### Advantageous Effects of Invention

According to the present invention, it is possible to provide a nonhuman animal that is suitably used for developing a novel drug using safety evaluation or drug efficacy evaluation specialized for humans, and developing a treatment method specific for a disease, a method for producing the nonhuman animal, and a drug evaluation method.

### BRIEF DESCRIPTION OF DRAWINGS

FIG. 1 A view showing an example of a method for producing a nonhuman animal according to the present embodiment.

FIG. 2 A view showing an example of a drug evaluation method according to the present embodiment.

FIG. 3 A view showing a series of steps of a neonate transplantation method in Experimental Example 1.

FIG. 4 A result obtained by performing single-cell analysis on chimeric nephrons formed from precursor cells transplanted into a subcapsular region of the kidney of a neonatal mouse in Experimental Example 1.

FIG. 5 A result of examining a proportion of foreign nephrons in a cell injection region by immunostaining 2 weeks after the transplantation in Experimental Example 1.

FIG. 6 A result obtained by intraperitoneally administering cisplatin to a mouse individual having a chimeric kidney 2 weeks after the transplantation in Experimental Example 2, recovering regenerated nephrons 48 hours later, and performing evaluation by immunofluorescence staining.

FIG. 7 A result obtained by intraperitoneally administering cisplatin to a mouse individual having a chimeric kidney 2 weeks after the transplantation in Experimental Example 2, recovering regenerated nephrons 48 hours later, and performing single-cell RNA sequencing.

FIG. 8 A result obtained by recovering regenerated nephrons, 4 months after the transplantation, from a mouse individual having a chimeric kidney in Experimental Example 2, and performing evaluation by immunofluorescence staining.

FIG. 9 (A) A result obtained by examining foreign nephrons in a cell injection region 2 weeks after the transplantation of GFP-expressing human nephron precursor cells in Experimental Example 3 by immunostaining. (B) A result obtained by intraperitoneally administering cisplatin to a mouse individual having a chimeric kidney 2 weeks after the transplantation of GFP-expressing human nephron precursor cells, recovering regenerated nephrons 48 hours later, and performing evaluation by immunofluorescence staining.

### DESCRIPTION OF EMBODIMENTS

### <<Nonhuman Animal>>

In one embodiment of the present invention, there is provided a nonhuman animal having a chimeric organ including a foreign cell in a body thereof in one embodiment.

Examples of the nonhuman animal include cats, dogs, horses, monkeys, cows, sheep, pigs, goats, rabbits, hamsters, guinea pigs, rats, and mice. Among these, the rodents are preferable from the viewpoint of a record of drug evaluation. Examples of the rodents include hamsters, guinea pigs, rats, and mice, and the rats and the mice are preferable.

The chimeric organ is not particularly limited, and examples thereof include the liver, the cornea, the skin, the large intestine, the small intestine, the pancreas, the stomach, the muscle tissue, the heart, the lung, the esophagus, the bone marrow, the kidney, the spleen, the testis, and the ovary, and an organ to be evaluated in an animal experiment is preferable.

For example, it is known that the expression of transporters on the cell surface of proximal renal tubular epithelial cells that are the main seat of a drug-induced kidney injury is different between humans and rodents. This species difference has long been pointed out as a discrepancy between the results of animal experiments (preclinical trials) and clinical trials in drug discovery, and in fact, there is a report that about 30% of the safety tests in animal experiments are false negatives (limitations due to the species difference).

In addition, in a toxin addition experiment by a monolayer culture of immortalized human renal tubular epithelial cells, which is the most common in vitro experimental system, there is a problem of a change in properties, such as a loss of expression of transporters OAT1 and OAT3. In recent years, models for evaluating drug toxicity while maintaining the polarity of renal tubular cells using a 3D-cultured kidney organoid or a fluidic device, and the like have been proposed. However, there is a problem with in vitro systems, such as inability to reproduce physiological administration of a drug due to the lack of blood flow or urine flow, and inability to culture for a long period of time, thus making it impossible to evaluate chronic toxicity (limitations of in vitro systems).

In a case where the kidney organoid is transplanted into a rodent, it matures by attracting a host blood vessel, but the kidney organoid expands due to urine produced since it is not connected to the urine excretion route of the host, and thus, it cannot survive for a long period of time.

The foreign cell contained in the chimeric organ is not particularly limited, and examples thereof include a rodent cell, a human cell, a cell derived from an individual patient, a labeled cell, or a genome-edited cell, or a cell obtained by combining these.

The present invention relates to construction of, for example, a foreign kidney (chimeric kidney) that is incorporated into the kidney of a host by injecting foreign nephron precursor cells into the renal development region of a mouse.

In the construction of the chimeric kidney, examples of the foreign cell include fetal renal cells of a mouse (allogeneic) and a rat (xenogeneic), NPCs induced from mouse ES cells, and NPCs induced from human iPS cells. The host mouse is preferably a fetus or a neonate.

The nonhuman animal of the present invention is preferably a drug administration model. Such a nonhuman animal preferably has a chimeric organ with a human, and is suitably used as a preclinical trial model at the time of new drug development.

Even in a case where the proportion of the foreign cells in the chimeric organ is low, it is possible to evaluate the influence on a human organ after drug administration in an animal experiment by acquiring a single cell derived from the foreign cell from the chimeric organ and evaluating the single cell.

In addition, a genetically modified nonhuman animal including a system for conditionally removing at least a part of a target organ of a host, such as a host animal capable of inducing programmed cell death in a conditional manner, may be used in order to increase the proportion of the foreign cells in the chimeric organ.

Examples of the system include a system in which a DTA nonhuman animal that expresses diphtheria toxin A (DTA) in a Cre recombinase activity-dependent manner is mated with a Six2-CreERT2 nonhuman animal in which a CreERT2 gene is introduced downstream of the Six2 promoter, and tamoxifen is brought into contact with a target organ of the obtained descendant (see Fujimoto T, Yamanaka S. Generation of Human Renal Vesicles in Mouse Organ Niche Using Nephron Progenitor Cell Replacement System. 2020; 32(11): 108130).

The DTA nonhuman animal has a transcription termination sequence sandwiched between two loxP sequences upstream of a gene encoding diphtheria toxin A. Therefore, the diphtheria toxin A is not expressed as it is. However, in a case where the transcription termination sequence sandwiched between the two loxP sequences is removed by a Cre recombinase, the diphtheria toxin A is expressed.

CreERT2 is typically limited to the cytoplasm, and can migrate to the nucleus to be active only after exposure to tamoxifen. In the above-described example, this phenomenon is used.

For example, in a case where a DTA nonhuman animal is mated with a Six2-CreERT2 nonhuman animal that specifically expresses CreERT2 in the metanephric mesenchyme, a nonhuman animal that specifically expresses the diphtheria toxin A in the metanephric tissue appears among the obtained descendants. Furthermore, Six2 is a transcription factor that is specifically expressed in the metanephric mesenchyme. By using such a nonhuman animal, the proportion of the foreign cells in the metanephric tissue can be increased.

A major advantage of this system is that since DTA and CreERT2 are specifically expressed in the metanephric mesenchyme of a nonhuman animal, there is no concern about toxicity to foreign cells including human cells.

Next, the created chimeric kidney is subjected to short-term administration or repeated administration of a drug, and the toxicity is evaluated. Specifically, the reaction of the drug-exposed foreign cells derived from the chimeric kidney is quantitatively evaluated by methods such as single-cell RNA sequencing, immunostaining, laser microdissection, and quantitative PCR. In addition, the quantitative evaluation is not limited to the transcription analysis (transcriptomics), and multi-omics analysis including genome analysis (genomics), protein analysis (proteomics), metabolite analysis (metabolomics), or the like can also be used.

In preclinical trials using rodents, for example, by using mice having chimeric kidneys of humans and mice, it is possible to perform the evaluation under conditions closer to those of clinical trials than in a case of analyzing mouse kidneys having different transporter expression from human kidneys.

In addition, the cells to be transplanted can be genetically modified to create a nonhuman animal having a genetically modified chimeric organ. Examples of the genetically modified foreign cell include a genome-edited cell. Examination on a difference in reactivity to a drug of the chimeric organ including a foreign cell in which a transporter has been knocked out by genome edition can lead to drug discovery.

Moreover, the use of a nonhuman animal having a chimeric organ created using a disease-specific cell as the foreign cell can lead to elucidation of pathological conditions and development of treatment methods.

In addition, in the two-dimensional culture of mature renal tubular cells in an in vitro human renal cell culture system, phenotypic changes such as a loss of expression of cell transporters occur (see Jenkinson S E, Chung G W. The limitations of renal epithelial cell line HK-2 as a model of drug transporter expression and function in the proximal tubule. 2012; 464(6): 601-11). Even in a three-dimensional culture that creates kidney organoids from stem cells, it has not been possible to create sufficiently mature kidneys, and the expression of transporters necessary for evaluating kidney injury is insufficient (see Wu H, Uchimura K. Comparative Analysis and Refinement of Human PSC-Derived Kidney Organoid Differentiation with Single-Cell Transcriptomics. 2018; 23(6): 869-881. e8).

In all of these culture systems, in a case of verifying a kidney injury by adding a drug to a culture medium, both the luminal side and the basal membrane side of the renal tubule are exposed to the drug without distinction, and it is thus impossible to reproduce the pharmacokinetics in vivo.

In order to solve the problem, attempts have been made to expose renal tubular cells to a drug by disposing the renal tubular cells in a lumen shape using a microfluidic device, but it is still difficult to accurately reproduce the physiological behavior in which the drug passes through the blood vessels, is filtered in the glomerulus, and is excreted and reabsorbed in the renal tubules. Furthermore, the kidney cannot be maintained in the culture for a long period of time, which is inappropriate as a chronic evaluation model.

The present invention solves the above-described problems.

Moreover, for the in-vivo transplantation of a human kidney organoid, it is known that in a case where the organoid is transplanted into a rodent, the organoid draws in the host blood vessel and matures (see van den Berg C W, Renal Subcapsular Transplantation of PSC-Derived Kidney Organoids Induces Neo-vasculogenesis and Significant Glomerular and Tubular Maturation In Vivo. 2018; 10(3): 751-765). However, since the transplanted human kidney organoid is not connected to a urine excretion route of the host, it expands due to the produced urine and cannot survive for a long period of 2 months or more and will disappear.

In addition, the blood vessels supplying nutrients to the organoid are mainly veins, and the transplanted organoid cannot be matured to the same level as the original kidney (Ryan A R, England A R. Vascular deficiencies in renal organoids and ex vivo kidney organogenesis. 2021; 477:98-116).

In the present invention, it has been confirmed that the foreign cells differentiate into mature nephrons and are connected to the urinary tract of the host for a long period of 4 months or more, which is superior to organoid transplantation in this respect.

In addition, it has been reported that a chimeric kidney can be created by injecting foreign mouse or rat stem cells into mouse embryos. However, the chimeric formation rate in a case of using human iPS cells is extremely low (see Gafni O, Weinberger L. Derivation of novel human ground state naive pluripotent stem cells. 2013; 504(7479): 282-6). Furthermore, since there is a concern that human iPS cells may differentiate into neural and reproductive systems, there is a high technical and ethical hurdle in creating human chimeric kidneys. On the other hand, in the present invention, since human cells that have differentiated into renal precursor cells can be used, there is no concern about such ethical problems.

In addition, it takes more than one year to create a genetically modified mouse, and it takes more time and is more expensive to genetically modify a plurality of genes. In the present invention, a mouse having a genetically modified chimeric organ can be created early and at a low cost by subjecting the foreign cell to genetic modification in a case of creating a chimeric kidney.

In addition, attempts to perform in-vivo gene edition by systemic or local administration of a virus or the like carrying a target gene have been reported, but the kidney is difficult for a virus to reach due to the glomerular filtration barrier, making knock-in efficiency poor (see Rubin J D, Barry M A. Improving Molecular Therapy in the Kidney. Mol Diagn Ther. 2020; 24(4): 375-96). In the present invention, it is possible to efficiently perform genetic modification by selecting and culturing stem cells in a large scale after having been subjected to genome edition in advance, and then transplanting the stem cells to create a chimeric kidney.

Until now, there has been no technology for operating human kidneys that function in vivo in nonhuman animals at a preclinical trial stage, but research has been accelerated due to the improvement of iPS cell technology and genome editing technology, and research and development using chimeric technology have a great impact.

The nonhuman animal of the present invention is preferably a pathological model. For example, the pathological model can be created by using, as a foreign cell, a cell obtained by differentiating a disease-specific human iPS cell into a desired precursor cell. In addition, a predetermined disease cell may be used as the foreign cell by genome edition, or a human cell may be used as the foreign cell and a drug treatment may be performed to create the pathological model.

By using such a pathological model, it is possible to elucidate the pathological condition of diseases with high unmet medical needs, such as a drug-induced kidney injury and a hereditary kidney disease, and to develop a treatment method therefor.

### <<Method for Producing Nonhuman Animal>>

In one embodiment of the present invention, there is provided a method for producing the above-described nonhuman animal, the production method including a step of injecting a foreign cell into a kidney of a fetus or a neonate.

The present inventors had invented a fetal transplantation method in which a mouse individual having a chimeric kidney is produced and survives by directly injecting foreign renal precursor cells into the retroperitoneal region of a fetal mouse in the mother body (Yamanaka S, Saito Y, Fujimoto T, Takamura T, Tajiri S, Matsumoto K, et al. Kidney Regeneration in Later-Stage Mouse Embryos via Transplanted Renal Progenitor Cells. J Am Soc Nephrol. 2019; 0(12): 2293-305).

This method has a low accuracy in the cell injection site and a low survival rate of the host fetus, and thus, a simpler method is required to establish the method as an evaluation model. Therefore, the present inventors have invented a neonatal transplantation method in which a foreign cell is directly injected into a subcapsular region of the kidney of a neonatal mouse aged 0 to 1 day after birth by utilizing a fact that nephron precursor cells remain until about 3 days after birth and renal development continues in mice.

Examples of the foreign cell to be injected into the kidney of a host neonate are the same cells as those described in <<Nonhuman Animal>>. In a case of creating a pathological model, it is preferable to use precursor cells obtained by differentiation of pluripotent stem cells derived from a patient with a hereditary disease or precursor cells in which at least one target gene has been edited by genome edition.

By using such precursor cells, it is possible to create a chimeric organ in 2 to 3 weeks, and it is possible to easily change the phenotype of the organ. According to the present invention, it is possible to easily produce a nonhuman animal having a chimeric organ without depending on the creation of a genetically modified nonhuman animal such as a knockout mouse. In addition, while the organoid is maintained in vivo for about 2 months, the chimeric organ in the nonhuman animal produced according to the present invention is maintained for a long period of time. Therefore, the nonhuman animal of the present invention can withstand repeated administration tests.

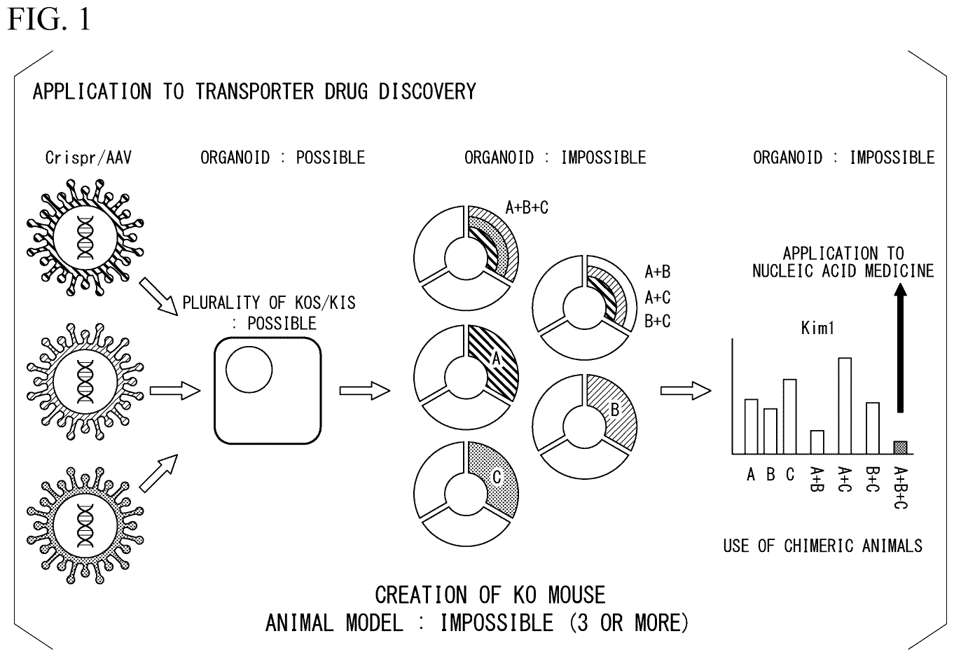

Moreover, the foreign cell is preferably a cell having a labeled protein associated with the edited target gene. As shown in FIG. 1, for example, a genome editing system such as a plurality of types of CRISPR-Cas systems that modify the functions of a plurality of types of renal tubule transporters, which are causative genes of a drug-induced kidney injury, is introduced into a foreign cell. In this case, a vector (for example, an adeno-associated virus vector: AAV) equipped with the CRISPR-Cas system is also equipped with a gene encoding a labeled protein associated with a target gene. For example, a virus vector equipped with a CRISPR-Cas system that modifies each of three target genes A, B, and C is equipped with a gene encoding a red fluorescent protein, a gene encoding a yellow fluorescent protein, and a gene encoding a green fluorescent protein, and the virus vector is infected into a precursor cell as a foreign cell in a combination of A, B, C, A+B, A+C, B+C, and A+B+C. Using a nonhuman animal having a chimeric organ produced using each of these foreign cells, combinations of genetic modifications that reduce nephrotoxicity are searched. The details of the optimum combination can be easily recognized from the type of the labeled fluorescence. This can be applied to drug discovery, leading to the development of a specific treatment method for a drug-induced kidney injury using a nucleic acid medicine or a low-molecular-weight compound.

On the other hand, for the genome-edited animal, a change in the phenotype by editing three or more genes requires a great deal of effort and time, which is thus unsuitable for searching for combinations.

### <<Drug Evaluation Method>>

In one embodiment of the present invention, there is provided a drug evaluation method including a step of administering a drug to the above-described nonhuman animal, and a step of acquiring a single cell derived from a foreign cell from a chimeric organ after the drug administration, and evaluating the single cell.

In the step of administering a drug to a nonhuman animal, the administration method is not particularly limited, and examples thereof include intranasal, transbronchial, intramuscular, transdermal, or oral methods, in addition to intra-arterial injection, intravenous injection, subcutaneous injection, and the like, and the administration method is preferably determined based on the administration method assumed in drug development.

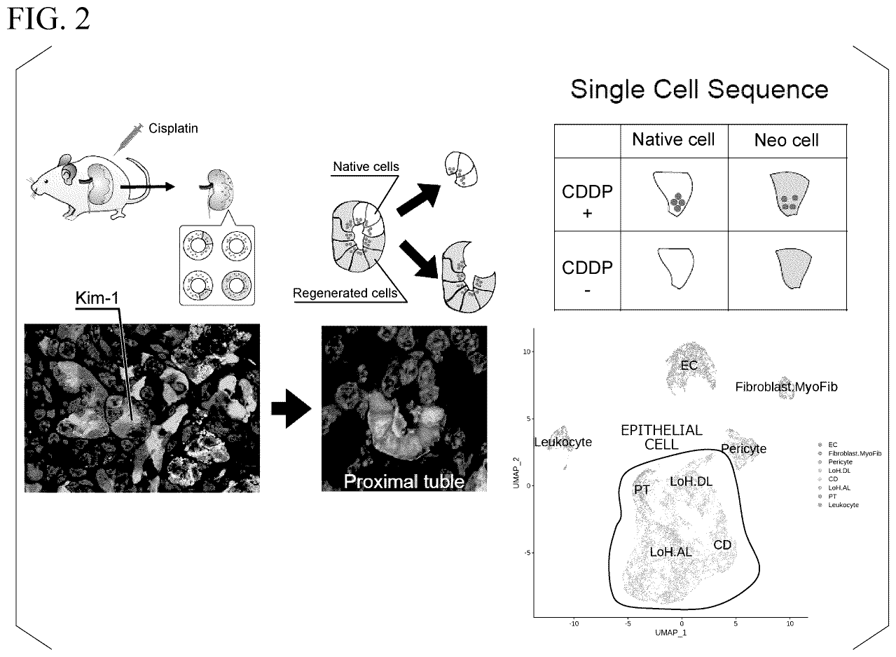

Next, a single cell derived from the foreign cell is acquired from the chimeric organ exposed to the drug after the drug administration, and the single cell is evaluated. The evaluation method is not particularly limited, and examples thereof include methods such as single-cell RNA sequencing, immunostaining, laser microdissection, and quantitative PCR. In order to acquire and evaluate a single cell derived from the foreign cell from the chimeric organ, an occupancy rate of the foreign cell in the chimeric organ may be low. In addition, it is possible to simultaneously evaluate a difference in sensitivity to the drug between the foreign cell and the host-derived cell. For example, as shown in FIG. 2, cisplatin (CDDP) is administered to a mouse having a chimeric kidney, and the presence or absence of a drug in each of a host-derived cell (native cell) and a foreign cell (Neo-cell) in a renal tubule can be evaluated with a single cell.

Moreover, by using an iPS cell derived from a patient as the foreign cell and evaluating the efficacy and safety of a drug using the created nonhuman animal, it is possible to make a contribution to tailor-made medical care.

### EXAMPLES

The present invention will be described below with reference to Examples, but is not limited to the following Examples.

### Experimental Example 1

The present inventors had succeeded in producing and surviving a mouse individual having a chimeric kidney by directly injecting foreign renal precursor cells into a retroperitoneal region of a fetal mouse in the mother body (Yamanaka S, Saito Y, Fujimoto T, Takamura T, Tajiri S, Matsumoto K, et al. Kidney Regeneration in Later-Stage Mouse Embryos via Transplanted Renal Progenitor Cells. J Am Soc Nephrol. 2019; 30(12): 2293-305).

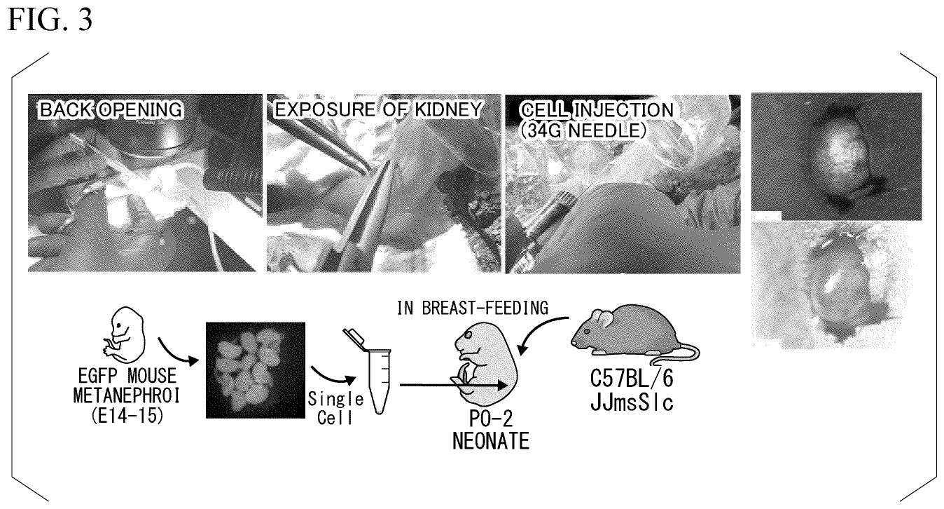

This method has a low accuracy in the cell injection site and a low survival rate of the host fetus, and thus, a simpler method is required to establish the method as an evaluation model. Therefore, the collected cells derived from the EGFP mouse embryonic E14 kidney were directly injected into a subcapsular region of the kidney of a neonatal mouse aged 0 to 1 day after birth (see FIG. 3) by utilizing a fact that nephron precursor cells remain until about 3 days after birth and the renal development continues in mice (see Hartman H A, Lai H L. Cessation of renal morphogenesis in mice. 2007; 310(2): 379-87).

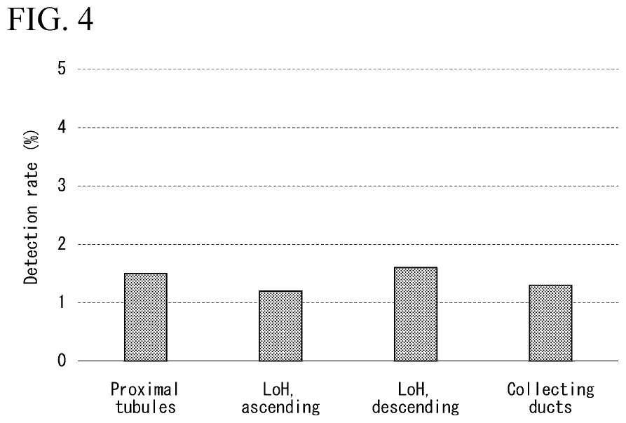

Single-cell analysis was performed on the chimeric nephrons formed from the precursor cells transplanted into the subcapsular region of the kidney of the neonatal mouse (see FIG. 4). As shown in FIG. 4, it was confirmed that the foreign cell was differentiated into a mature renal tubule.

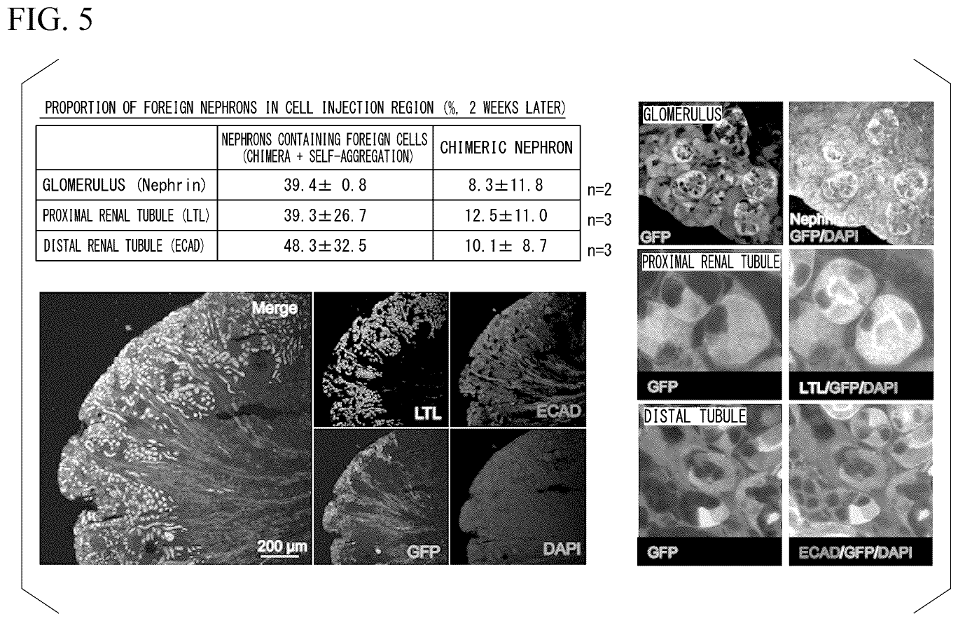

Furthermore, a proportion of the foreign nephrons in the cell injection region 2 weeks after the transplantation was examined by immunostaining (see FIG. 5). As shown in FIG. 5, the formation of a chimeric glomerulus and a proximal to distal renal tubule was confirmed. It was confirmed that the foreign cells express a marker of the mature renal tubule.

### Experimental Example 2

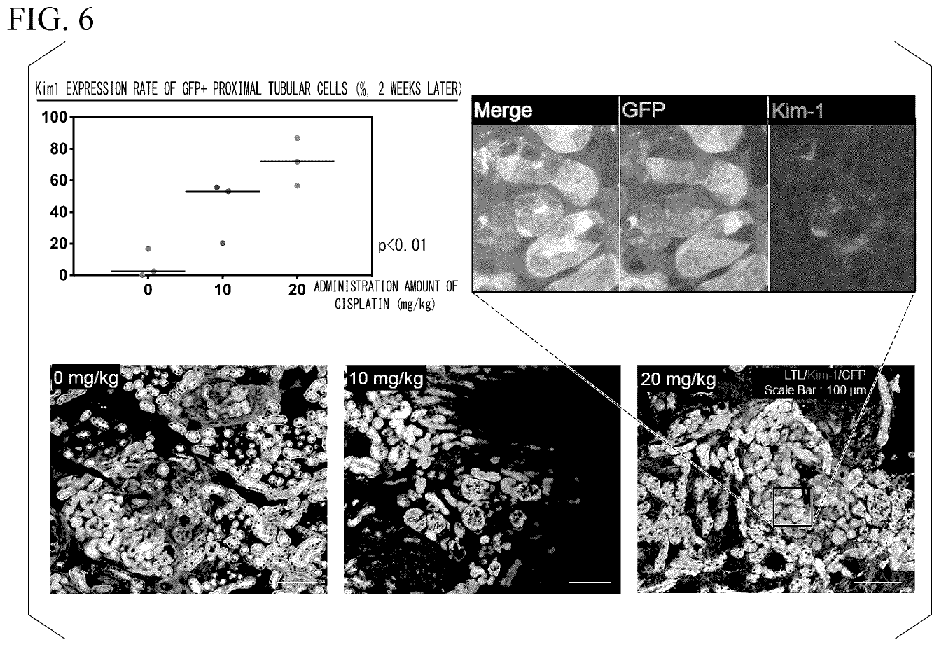

Cisplatin was intraperitoneally administered to a mouse individual having a chimeric kidney 2 weeks after transplantation, which was created in Experimental Example 1, and the regenerated nephrons were recovered 48 hours later and evaluated by immunofluorescence staining. As shown in FIG. 6, a dose-dependent expression of Kim-1 was confirmed in the foreign proximal renal tubular cells by the administration of cisplatin.

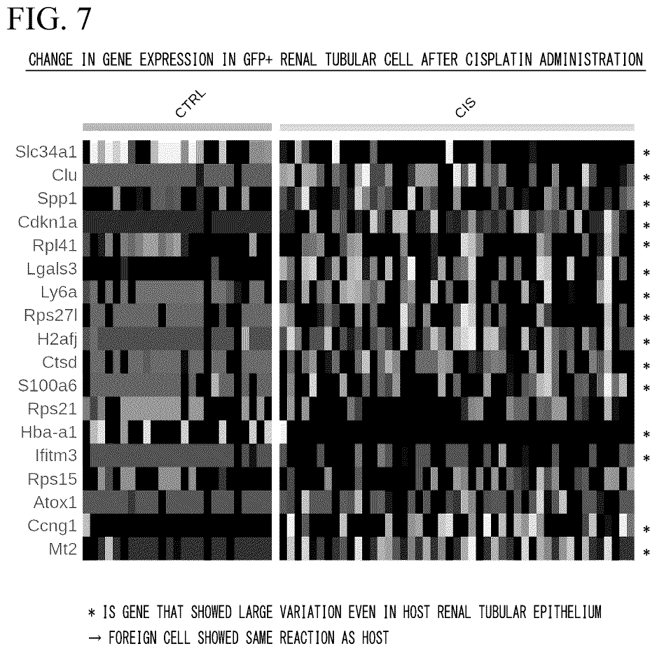

In addition, single-cell RNA sequencing was performed on the recovered sample. As shown in FIG. 7, the enhanced expression of disorder-related genes other than Kim-1 was also confirmed.

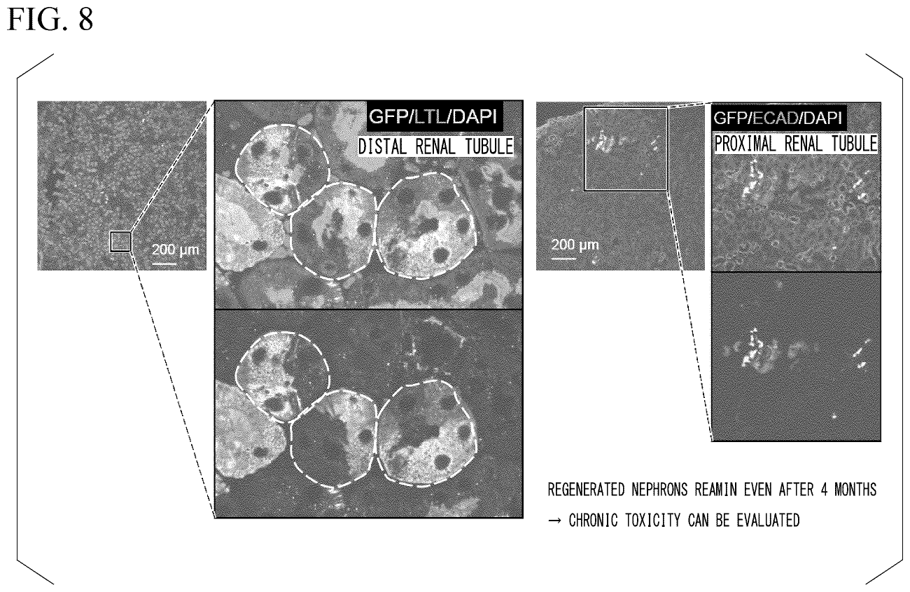

In the mouse individual having a chimeric kidney, which was created in Experimental Example 1, regenerated nephrons were recovered 4 months after the transplantation, and evaluated by immunofluorescence staining. As shown in FIG. 8, the regenerated nephrons remained even 4 months after the transplantation. From this, it was confirmed that chronic repeated administration tests are also possible by taking advantage of a fact that the mouse individual having the chimeric kidney survives for a long period of time.

### Experimental Example 3

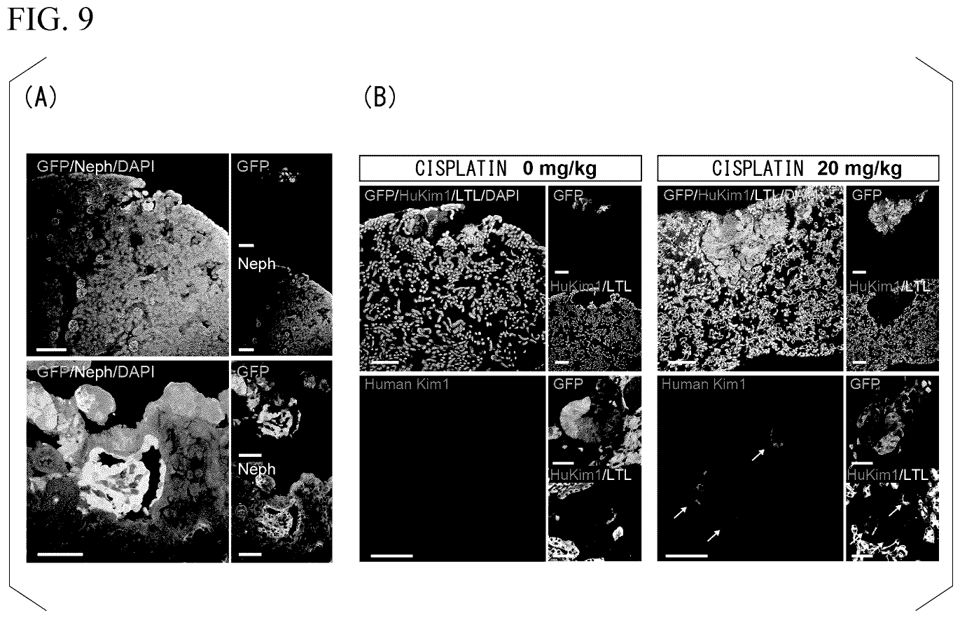

In Experimental Example 1, instead of the collected cells derived from the EGFP mouse embryonic E14 kidney, GFP-expressing human nephron precursor cells were directly injected into a subcapsular region of the kidney of a neonatal mouse aged 0 to 1 day after birth. The foreign nephrons in the cell injection region 2 weeks after the transplantation were examined by immunostaining (see (A) of FIG. 9). As shown in (A) of FIG. 9, the formation of glomeruli and renal tubules derived from GFP-expressing human nephron precursor cells was confirmed.

In addition, cisplatin was intraperitoneally administered to a mouse individual having a chimeric kidney 2 weeks after the transplantation of the GFP-expressing human nephron precursor cells, and the regenerated nephrons were recovered 48 hours later and evaluated by immunofluorescence staining. As shown in (B) of FIG. 9, an expression of human Kim-1 was confirmed in the foreign proximal renal tubular cells by the administration of cisplatin.

### INDUSTRIAL APPLICABILITY

According to the present invention, it is possible to provide a nonhuman animal that is suitably used for developing a novel drug and developing a treatment method specific for a disease, a method for producing the nonhuman animal, and a drug evaluation method.

1. A nonhuman animal comprising:
a chimeric organ including a foreign cell in a body of the nonhuman animal.

2. The nonhuman animal according to claim 1,
wherein the foreign cell is a rodent cell, a human cell, a cell derived from an individual patient, a labeled cell, a genome-edited cell, or a cell obtained by combining these cells.

3. The nonhuman animal according to claim 1,
wherein the nonhuman animal is a pathological model.

4. The nonhuman animal according to claim 1,
wherein the nonhuman animal is a drug administration model.

5. A method for producing the nonhuman animal according to any one of claims 1 to 4, the production method comprising:
a step of injecting a foreign cell into a kidney of a fetus or a neonate.

6. The production method according to claim 5,
wherein the foreign cell is a precursor cell obtained by differentiating a pluripotent stem cell derived from a hereditary disease patient.

7. The production method according to claim 5,
wherein the foreign cell is a cell in which at least one target gene is edited.

8. The production method according to claim 7,
wherein the foreign cell is a cell having a labeled protein associated with the edited target gene.

9. A drug evaluation method comprising:
a step of administering a drug to the nonhuman animal according to any one of claims 1 to 4; and
a step of acquiring a single cell derived from a foreign cell from a chimeric organ after the drug administration and evaluating the single cell.
